(19) United States
(12) Patent Application Publication
(10) Pub. No.: US 2008/0166408 A1
(43) Pub. Date: Jul. 10, 2008

(54) Oral Dosage Form Comprising Rosiglitazone

(76) Inventors: Joanne Heafield, Essex (GB); Vincenzo Re, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/815,326

(22) PCT Filed: Feb. 3, 2006
(86) PCT No.: PCT/EP2006/000997

§ 371 Date: Aug. 2, 2007

(30) Foreign Application Priority Data
Feb. 7, 2005 (GB) 0502475.7

(51) Int. Cl. A61K 9/24; A61K 31/4436; A61P 3/10; A61P 25/00; A61P 17/00; A61P 9/00

(52) U.S. Cl. 424/472; 514/342

(57) ABSTRACT

An oral dosage form, such as a bilayer tablet, comprising a first layer of a first composition and a second layer of a second composition, each composition comprising 5-[4-[2-(N-methyl-N-(2 pyridyl)amino)ethoxy]benzyl]thiazolidine-2,4-dione or a pharmaceutically acceptable salt or solvate thereof, (‘the drug’) and a pharmaceutically acceptable carrier therefor, wherein the first and second compositions are arranged to release drug at differing release rates on administration; a process for preparing such a dosage form; and the use of such a dosage form in medicine.

The present invention relates to an oral dosage form comprising 5-[4-[2-(N-methyl-N-(2 pyridyl)amino)ethoxy]benzyl]thiazolidine-2,4-dione (hereinafter ‘Compound A’) or a pharmaceutically acceptable salt or solvate thereof, to a process for preparing such a dosage form and to the use of such a dosage form in medicine.

The use of a coating to control the rate of release of an active agent has received considerable attention and many different devices have been developed for such a purpose. For example, International Patent Application, Publication Number WO 01/05430 describes a drug delivery device that enables the delivery of drug substances which exhibit pH dependent solubility, in particular compounds that are more soluble at low pH levels (less than pH 2) than at near neutral pH levels (greater than about pH 5). Such delivery devices are characterised by the presence of a coating that is impermeable and insoluble in the fluid of the environment of use.

International patent application, Publication Number WO 95/30422 describes a series of controlled-release dosage forms of azithromycin. In particular, there is described a series of dosage forms that reduce the exposure of the upper GI tract (e.g. the stomach) to high concentrations of azithromycin, by the use of a pH dependent coating. Such dosage forms do not feature openings through which release of the drug substance may occur.

U.S. Pat. No. 6,099,859 describes a controlled release tablet for the delivery of an antihyperglycaemic drug, which comprises an osmotically active drug-containing core and a semipermeable membrane, wherein the semipermeable membrane is permeable to the passage of water and biological fluids and is impermeable to the passage of the drug substance. The semipermeable membrane contains at least one passageway for the release of the antihyperglycaemic drug.

U.S. Pat. No. 5,543,155 describes a diffusion-osmotic controlled drug release pharmaceutical composition comprising a one- or two-layer tablet core containing hydroxypropyl methylcellulose, said core having a film-coat comprising an ammonium methacrylate copolymer.

Additional devices that utilise a coating to control the rate of release of an active agent are discussed in U.S. Pat. No. 5,004,614. This patent describes a tablet core provided with an outer coating that is substantially impermeable to environmental fluid. The said outer coating may be prepared from materials that are either insoluble or soluble in the environmental fluids. Where a soluble material is used, the coating is of sufficient thickness that the core is not exposed to environmental fluid before the desired duration of the controlled release of the active agent has passed. Through this impermeable outer coating, one or more opening(s) has been created, so as to provide environmental fluids with an access route to the core. Therefore, upon ingestion of the coated tablet, gastro-intestinal fluid can enter the opening(s) and contact or penetrate the core, to release the active agent. The result is that the active agent is released in a controlled manner out of the opening(s) only. The preferred geometry is such that there is a circular hole on the top and bottom face of the coated tablet. The opening(s) in question have an area from about 10 to 60 percent of the face area of the coated tablet. The rate of drug release is found to be directly related to the diameter of the opening(s) and to the solubility of the matrix core and active agent, allowing the possibility for a variety of drug release profiles be it zero or first order release.

The substantially impermeable coatings of U.S. Pat. No. 5,004,614 are not suitable for the controlled release of all active agents, especially pharmaceutically active weak bases or pharmaceutically acceptable salts and solvates thereof. Such active agents exhibit a marked pH dependent solubility, i.e. they are more soluble at around pH 2, associated with regions found in the stomach, compared to their solubility in the generally neutral conditions of the small intestine, around pH 7.

International Patent Application, Publication Number WO 03/068195 discloses an oral dosage form comprising an erodable core which contains a pharmaceutically active weak base or a pharmaceutically acceptable salt or solvate thereof, such as Compound A, the core having a coating with one or more openings leading to the core, and the coating being erodable under predetermined pH conditions. This provides a beneficial means for administration of a pharmaceutically active weak base or a pharmaceutically acceptable salt or solvate thereof, such as Compound A, where it is desirable that release of the active compound takes place in more than one pH environment, based on the finding that it is also beneficial for the coating to be erodable or soluble in a pH dependent manner.

European Patent Application, Publication Number 0 306 228 A1 relates to certain thiazolidinedione derivatives disclosed as having antihyperglycaemic and hypolipidaemic activity. One particular thiazolidinedione disclosed in EP 0 306 228 A1 is Compound A. International Patent Application, Publication Number WO 94/05659 discloses certain salts of Compound A including the maleate salt at Example 1 thereof. Compound A or a pharmaceutically acceptable salt thereof or a pharmaceutically acceptable solvate thereof, may be prepared using known methods, for example those disclosed in EP 0 306 228 and WO 94/05659. The disclosures of EP 0 306 228 and WO 94/05659 are incorporated herein by reference.

Compound A and pharmaceutically acceptable salts or solvates thereof have useful pharmaceutical properties. In particular, Compound A or a salt or solvate thereof is indicated to be useful for the treatment and/or prophylaxis of diabetes mellitus, conditions associated with diabetes mellitus and certain complications thereof; Alzheimer's Disease, mild cognitive impairment, psoriasis, asthma, atherosclerosis, metabolic syndrome, impaired glucose tolerance and impaired fasting glucose.

International Patent Application, Publication Number WO 00/28990 describes various modified release pharmaceutical compositions comprising insulin sensitisers, including Compound A and pharmaceutically acceptable salts or solvates thereof.

International Patent Application, Publication Number WO 00/28990 describes a method of treating Type 2 diabetes mellitus and conditions associated with diabetes mellitus, using certain pharmaceutical compositions, including modified release compositions, which provide a Threshold Plasma Concentration of Compound A or a pharmaceutically acceptable salt or solvate thereof.

International Patent Application Number PCT/EP2004/008843 (WO 05/013935) describes an oral dosage form comprising a first composition and a second composition, each composition comprising a pharmaceutically acceptable weak base, especially Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH.

Compound A is a pharmaceutically acceptable weak base.

Compound A and pharmaceutically acceptable salts or solvates thereof, in particular the maleate salt, have been found to exhibit marked pH dependent solubility, i.e. they are more soluble in the acidic conditions of the stomach (around pH 2) than in the near neutral conditions of the lower intestine (around pH 7).

It is an object of the present invention to provide an oral dosage form comprising Compound A or a pharmaceutically acceptable salt or solvate thereof, which provides a maximised beneficial effect, for example on glycaemic control, for an extended period of time. Such a dosage form is considered to be suitable for once daily administration. Such a dosage form is also indicated for administration in both fasted and fed states, with substantially no clinically relevant food effect.

The present invention is based on the finding that one or more objects of the invention can be accomplished by means of a bilayer oral dosage form in which the layers are arranged to release drug at differing release rates on administration, and the further finding that this can be accomplished while avoiding use of a release-controlling coating.

Accordingly, the present invention provides an oral dosage form comprising a first layer of a first composition and a second layer of a second composition, each composition comprising Compound A or a pharmaceutically acceptable salt or solvate thereof (‘the drug’) and a pharmaceutically acceptable carrier therefor, wherein the first and second compositions are arranged to release drug at differing release rates on administration.

Suitably the rate of release of the drug from the dosage form is substantially independent of pH.

Suitably, the release rate of the drug from the first composition is substantially greater than from the second composition. It is envisaged that, the first composition is an immediate release composition. It is also envisaged that, the second composition is a modified release composition.

In one aspect, the first composition is arranged so that in use it releases substantially all of the Compound A or a pharmaceutically acceptable salt or solvate thereof, in the stomach.

In a further aspect, the second composition is arranged so that in use it releases substantially all of the Compound A or a pharmaceutically acceptable salt or solvate thereof in the small intestine.

Suitably, the dosage form is a tablet form.

In one aspect the oral dosage form is arranged to release Compound A or a pharmaceutically acceptable salt or solvate thereof, such that the mean maximum plasma level concentration (“Cₘₐₓ”) value of the drug is maintained substantially independent of food during use, i.e. the observed Cₘₐₓ value is substantially similar in both fasted and fed states during use.

In another aspect the oral dosage form is arranged to release Compound A or a pharmaceutically acceptable salt or solvate thereof, such that the mean area under the plasma concentration versus time curve over the dosing interval at steady state (“AUC”) is maintained substantially independent of food during use, i.e. the observed AUC is substantially similar in both fasted and fed states during use.

Thus, in a preferred aspect in operation the oral dosage form releases Compound A or a pharmaceutically acceptable salt or solvate thereof, so that both the Cₘₐₓ value and AUC observed on administration are maintained substantially independent of food during use, i.e. the observed Cₘₐₓ value and AUC are substantially similar in both fasted and fed states during use.

Suitably, the first composition is formulated so that it provides immediate release of Compound A or a pharmaceutically acceptable salt or solvate thereof, on contact with aqueous media. Suitably, the second composition is formulated so that it provides modified release of Compound A or a pharmaceutically acceptable salt or solvate thereof, on contact with aqueous media.

The compositions can be formed in any shape or mutual conformation providing the required objective of the invention is met but generally each composition comprises a single layer of drug.

Most suitably, the dosage form is formulated so as to release drug to substantially the same extent in both the stomach and the intestines, i.e. the dosage form is formulated to compensate for the pH dependency of Compound A.

The desired release profile of the oral dosage form is achievable without the enteric coatings or apertured impermeable or pH dependent coatings used in the prior art acknowledged above.

However, as a protection for the dosage form, to prevent contamination before dosing, it may desirable to provide a conventional seal coating to the dosage form.

According to yet a further aspect of the present invention, there is provided a process for preparing an oral dosage form which dosage form comprises a first composition and a second composition, each composition comprising Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH;

which process comprises the steps of sequentially or simultaneously:
(i) formulating the drug into the first composition; and
(ii) formulating the drug into the second composition; and the steps of sequentially or simultaneously:
(i) forming the first composition into a first layer; and
(ii) forming the second composition into a second layer; and
(iii) combining the layers into a multilayer, especially a bilayer dosage form,
whereby the first and second layers are formulated to release drug at differing release rates on administration such that the rate of release of the drug from the dosage form is substantially independent of pH.

The first and second compositions may be prepared by compressing suitable ingredients in conventional manner to form a compacted mass in multiple layers, which comprises the dosage form, typically as a tablet. The dosage form may be prepared using conventional tablet excipients and formulation compression methods. Thus, the dosage form typically comprises the active agent or agents along with excipients that impart satisfactory processing and compression characteristics such as diluents, binders and lubricants. Additional excipients that may form part of the dosage form include disintegrants, flavourants, colorants, release modifying agents and/or solubilising agents such as surfactants, pH modifiers and complexation vehicles. Typically, the active agent and excipients are thoroughly mixed prior to compression into a multilayer, especially a bilayer, tablet. The dosage form may be formed by wet granulation methods, dry granulation methods or by direct compression. The dosage form may be produced according to any desired pre-selected shape such as bi-convex, hemi-spherical, near hemispherical, round, oval, generally ellipsoidal, oblong, generally cylindrical or polyhedral, e.g. a triangular prism shape. The term “near hemi-spherical” is intended to be construed in the manner described in U.S. Pat. No. 5,004,614. Suitably, the dosage form is formulated into a bi-convex shape, e.g. having two domed opposite surfaces.

As indicated above, the oral dosage form of the present invention is considered to be suitable for once daily administration and during use is indicated to provide a therapeutic effect over an extended period of time, such as up to 24 hours, for example, up to 12, 14, 16, 18, 20 and 24 hours per unit dose.

As used herein, the term “modified release” means a composition which has been designed to produce a desired pharmacokinetic profile by choice of formulation. Modified release also includes modified release compositions in combination with non-modified release compositions. For example, the term “modified release” shall comprise delayed, pulsed and sustained release either alone or in any combination.

In one aspect the modified release composition provides delayed release of Compound A or a pharmaceutically acceptable salt or solvate thereof. Delayed release is conveniently obtained by use of a gastric resistant formulation such as an enteric formulation.

In a further aspect the modified release composition provides sustained release of Compound A or a pharmaceutically acceptable salt or solvate thereof, for example providing release of the active agent over a time period of up to 26 hours, up to 24 hours, up to 18 hours, or up to 16 hours; suitably in the range of 4 to 24 hours; preferably in the range of 12 to 24 hours.

Sustained release is typically provided by use of a sustained release matrix, such as disintegrating, non-disintegrating or eroding matrices.

Sustained release is suitably obtained by use of a non-disintegrating matrix tablet formulation. Suitable non-disintegrating matrix tablet formulations are provided by the incorporation of methacrylates, cellulose acetates, carbomers and hydroxypropyl methylcellulose phtahlate into the tablet. Examples of suitable materials include Eudragit RS™ (Poly(ethyl acrylate, methyl methacrylate, trimethylammonioethyl methacrylate chloride) 1:2:0.1), Eudragit RL™ (Poly(ethyl acrylate, methyl methacrylate, trimethylammonioethyl methacrylate chloride) 1:2:0.2), Carbopol 971P™ (carbomer), HPMCP-HP-55S™ (hydroxypropyl methylcellulose phthalate).

Sustained release is further obtained by use of a disintegrating matrix tablet formulation, for example by incorporating methacrylates, methylcellulose or hydroxypropyl methylcellulose into the tablet. Examples of suitable materials include Eudragit L™ (Poly(methacrylic acid, ethyl acrylate) 1:1) and Methocel K4M™ (hydroxypropyl methylcellulose).

In yet a further aspect the modified release composition provides pulsed release of Compound A or a pharmaceutically acceptable salt or solvate thereof, for example providing up to 4, for example 2, pulses of active agent per 24 hours.

Suitable materials for an immediate release composition, such as the first composition, include saccharoses, for example lactose and maltose. Most suitably, the immediate release composition is predominantly lactose. More suitably, the immediate release composition consists essentially of lactose and magnesium stearate.

A suitable dosage range for Compound A or a pharmaceutically salt or solvate thereof is up to 12 mg, for example, 1 to 12 mg. Thus, suitable dosage forms comprise 1, 2, 3, 4, 5, 6, 7, 8, 9, 10, 11 or 12 mg of Compound A or a pharmaceutically acceptable salt or solvate thereof.

Particular dosage forms comprise 2 to 4 mg of Compound A or a pharmaceutically acceptable salt or solvate thereof.

Particular dosage forms comprise 4 to 8 mg of Compound A or a pharmaceutically acceptable salt or solvate thereof.

Particular dosage forms comprise 8 to 12 mg of Compound A or a pharmaceutically acceptable salt or solvate thereof.

One dosage form comprises 1 mg of Compound A or a pharmaceutically acceptable salt or solvate thereof.

One dosage form comprises 2 mg of Compound A or a pharmaceutically acceptable salt or solvate thereof.

Preferred dosage forms comprise 4 mg of Compound A or a pharmaceutically acceptable salt or solvate thereof.

Preferred dosage forms comprise 8 mg of Compound A or a pharmaceutically acceptable salt or solvate thereof.

The amount of Compound A or a pharmaceutically acceptable salt or solvate thereof present in the first composition and the second composition may be varied in accordance with the desired dissolution profile.

For example, where the oral dosage form comprises 8 mg of Compound A or a pharmaceutically acceptable salt or solvate thereof, the dosage form suitably comprises a layer comprising 1 mg of Compound A or a pharmaceutically salt or solvate thereof, and a layer comprising 7 mg of Compound A or a pharmaceutically salt or solvate thereof. Alternatively, the dosage form may comprise a layer comprising 4 mg of Compound A or a pharmaceutically salt or solvate thereof, and a layer comprising 4 mg of Compound A or a pharmaceutically salt or solvate thereof. More suitably, the dosage form comprises a layer comprising 2 mg of Compound A or a pharmaceutically salt or solvate thereof, and a layer comprising 6 mg of Compound A or a pharmaceutically salt or solvate thereof. Preferably, the dosage form comprises a layer comprising 3 mg of Compound A or a pharmaceutically salt or solvate thereof, and a layer comprising 5 mg of Compound A or a pharmaceutically salt or solvate thereof.

Where the oral dosage form comprises 2 mg of Compound A or a pharmaceutically acceptable salt or solvate thereof, the dosage form suitably comprises a layer comprising 0.75 mg of Compound A or a pharmaceutically salt or solvate thereof, and a layer comprising 1.25 mg of Compound A or a pharmaceutically salt or solvate thereof.

Where the oral dosage form comprises 4 mg of Compound A or a pharmaceutically acceptable salt or solvate thereof, the dosage form suitably comprises a layer comprising 1.5 mg of Compound A or a pharmaceutically salt or solvate thereof, and a layer comprising 2.5 mg of Compound A or a pharmaceutically salt or solvate thereof.

By adjustment of the release rates of the first and second compositions, and adjusting the other variables mentioned above and the surface area of the dosage form, the release rates in the different environmental conditions can be harmonised to obtain comparable release rates under different body environments, and so achieve more constant dosing to a patient.

Preferably the dissolution rates of the oral dosage forms of this invention are arranged so that the rate of release is substantially similar in the different pH environments experienced by the dosage form on administration. Dissolution rates may be assessed by in vitro testing in solutions of the appropriate pHs. For example, when comparing dissolution in the stomach and intestine, tests may be carried out initially at pH 1.5 with a transfer to pH 6.8 after 2 hours or 4 hours, as an assumed time for residence in the stomach before emptying into the intestines of a notional patient in respectively fasted and fed conditions.

As mentioned above, Compound A or a pharmaceutically acceptable salt or solvate thereof when administered in an oral dosage form of this invention is indicated to be useful for the treatment and/or prophylaxis of diabetes mellitus, conditions associated with diabetes mellitus and certain complications thereof; Alzheimer's Disease, mild cognitive impairment, psoriasis, asthma, atherosclerosis, metabolic syndrome, impaired glucose tolerance and impaired fasting glucose (hereinafter referred to as the ‘Disorders of the Invention’). Suitably, Compound A or a pharmaceutically acceptable salt or solvate thereof when administered in an oral dosage form of this invention is indicated to be useful in the treatment and/or prophylaxis of diabetes mellitus, conditions associated with diabetes mellitus and certain complications thereof. Suitably, Compound A or a pharmaceutically acceptable salt or solvate thereof when administered in an oral dosage form of this invention is indicated to be useful in the treatment and/or prophylaxis of Alzheimer's Disease. Suitably, Compound A or a pharmaceutically acceptable salt or solvate thereof when administered in an oral dosage form of this invention is indicated to be useful in the treatment and/or prophylaxis of mild cognitive impairment. Suitably, Compound A or a pharmaceutically acceptable salt or solvate thereof when administered in an oral dosage form of this invention is indicated to be useful in the treatment and/or prophylaxis of psoriasis. Suitably, Compound A or a pharmaceutically acceptable salt or solvate thereof is indicated to be useful in the treatment and/or prophylaxis of asthma. Suitably, Compound A or a pharmaceutically acceptable salt or solvate thereof when administered in an oral dosage form of this invention is indicated to be useful in the treatment and/or prophylaxis of atherosclerosis. Suitably, Compound A or a pharmaceutically acceptable salt or solvate thereof when administered in an oral dosage form of this invention is indicated to be useful in the treatment and/or prophylaxis of metabolic syndrome. Suitably, Compound A or a pharmaceutically acceptable salt or solvate thereof is indicated to be useful in the treatment and/or prophylaxis of impaired glucose tolerance. Suitably, Compound A or a pharmaceutically acceptable salt or solvate thereof when administered in an oral dosage form of this invention is indicated to be useful in the treatment and/or prophylaxis of impaired fasting glucose.

In a preferred embodiment the present invention provides a method for the treatment and/or prophylaxis of the Disorders of the Invention which method comprises administering an oral dosage form of this invention comprising Compound A or a pharmaceutically acceptable salt or solvate thereof, to a human or non-human mammal in need thereof.

In a further preferred embodiment the present invention provides an oral dosage form of the invention comprising Compound A or a pharmaceutically acceptable salt or solvate thereof for use in the treatment and/or prophylaxis of the Disorders of the Invention.

As used herein, the term “pharmaceutically acceptable” embraces compounds, compositions and ingredients for both human and veterinary use. For example the term “pharmaceutically acceptable salt” embraces a veterinarily acceptable salt. In particular, suitable pharmaceutically acceptable salted forms of Compound A include those described in European Patent Number 0 306 228 and International Patent Application, Publication Number WO 94/05659. A particularly preferred form of Compound A is the maleate salt.

Suitable pharmaceutically acceptable solvates include hydrates.

As used herein, the term “Cₘₐₓ” shall mean the mean maximum plasma level concentration.

As used herein the term “AUC” shall mean the mean area under the plasma concentration versus time curve over the dosing interval at steady state.

No adverse toxicological effects are indicated in the above mentioned treatments.

All publications, including but not limited to patents and patent applications, cited in this specification are herein incorporated by reference as if each individual publication were specifically and individually indicated to be incorporated by reference herein as though fully set forth.

In the following Examples, dosage forms were formed by conventional means by mixing together the active ingredients with excipients and compressing to form a multilayer dosage form. These Examples are intended to be by way of illustration rather than limitation of the present invention.

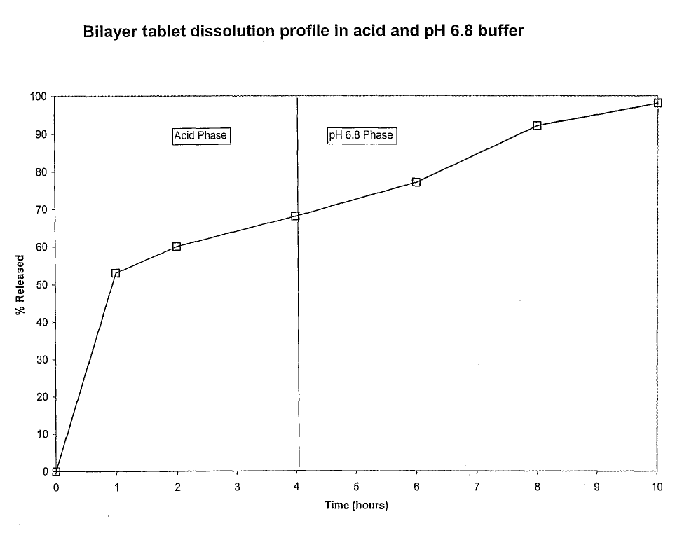

FIG. 1 is a graph of dissolution against time for an oral dosage form in accordance with Example 1 below.

### EXAMPLE 1

A bilayer tablet was formulated by combining layer (A) below to provide non-modified release (i.e. immediate) release of Compound (A) and layer (B) below to provide sustained release of Compound (A).

| | mg/tablet | |
|---|---|---|
| Layer A | | |
| Compound (A) as maleate | 3 | mg (pfb*) |
| Sodium Starch Glycollate | 1.500 | |
| Hypromellose | 1.500 | |
| Microcrystalline Cellulose | 6.000 | |
| Yellow Iron Oxide | 0.015 | |
| Magnesium Stearate | 0.250 | |
| Lactose Monohydrate to | 50 | |
| Layer B | | |
| Compound (A) as maleate | 5 | mg (pfb*) |
| Sodium Starch Glycollate | 2.500 | |
| Hypromellose | 2.500 | |
| Microcrystalline Cellulose | 10.000 | |
| Methacrylic Acid Copolymer, Type C | 50.000 | |
| Polyethylene Glycol | 20.000 | |
| Magnesium Stearate | 1.000 | |
| Lactose Monohydrate to | 150 | |

Additionally, immediate release layers, such as described in Example 1 may be combined with the modified release layers described below:

### EXAMPLE 2

### Sustained Release by Use of a Matrix Layer

A matrix layer is formed from the following mixture:

| | mg/tablet |
|---|---|
| Compound (A) | 5 (pfb*) |
| Eudragit L100-55 | 150 |
| Lactose monohydrate | 50 |
| Eudragit RS powder | to 500 |

### EXAMPLE 3

### Sustained Release by Use of a Mixed Eudragit Matrix Layer

A matrix layer is formed from the following mixture:

| | mg/tablet |
|---|---|
| Compound (A) | 5 (pfb*) |
| Eudragit L100-55 | 74 |
| Eudragit RS powder | 18.5 |
| Colloidal Silicon dioxide | 0.6 |
| Magnesium stearate | 1.5 |
| Lactose monohydrate | to 150 |

### EXAMPLE 4

### Sustained Release by Use of a Mixed Carbopol Matrix Layer

A matrix layer is formed from the following mixture:

| | mg/tablet |
|---|---|
| Compound (A) | 5 (pfb*) |
| Anhydrous dibasic calcium phosphate | 35.7 |
| Carbopol 971P | 22.5 |
| Carbopol 974P | 7.5 |
| Talc | 0.75 |
| Lactose monohydrate | to 150 |

*pfb = pure free base

1-15. (canceled)

16. An oral dosage comprising a first layer of a first composition and a second layer of a second composition, each composition comprising a drug, wherein the drug is 5-[4-[2-(N-methyl-N-(2 pyridyl)amino)ethoxy]benzyl]thiazolidine-2,4-dione, or a pharmaceutically acceptable salt or hydrate thereof, and a pharmaceutically acceptable carrier therefor, wherein the first and second compositions are arranged to release the drug at differing release rates on administration.

17. An oral dosage form according to claim 16, wherein the release rate of the drug from the first composition is substantially greater than from the second composition.

18. An oral dosage form according to claim 16, wherein the first composition is an immediate release composition.

19. An oral dosage form according to claim 16, wherein the second composition is a modified release composition.

20. An oral dosage form according to claim 16, wherein the first composition is arranged so that in use it releases substantially all of the drug in the stomach.

21. An oral dosage form according to claim 16, wherein the second composition is arranged so that in use it releases substantially all of the drug in the small intestine.

22. An oral dosage form according to claim 16, which dosage form is arranged to release the drug such that the mean maximum plasma level concentration value of the drug is maintained substantially independent of food during use.

23. An oral dosage form according to claim 16, which dosage form is arranged to release the drug such that the mean area under the plasma concentration versus time curve over the dosing interval at steady state is maintained substantially independent of food during use.

24. An oral dosage form according to claim 16, which dosage form is arranged to release the drug so that both the mean maximum plasma level concentration value and the mean area under the plasma concentration versus time curve over the dosing interval at steady state observed on administration are maintained substantially independent of food during use.

25. An oral dosage form according to claim 16, wherein the first composition is formulated so that it provides immediate release of the drug on contact with aqueous media.

26. An oral dosage form according to claim 16, wherein the second composition is formulated so that it provides modified release of the drug on contact with aqueous media.

27. An oral dosage form according to claim 16, wherein the dosage form is a tablet form.

28. An oral dosage form according to claim 27 in the form of a bilayer tablet comprising a first layer of an immediate release composition containing 3 mg (pfb) of the drug and a second layer of a modified release composition containing 5 mg (pfb) of the drug.

29. A process for preparing an oral dosage form which dosage form comprises a first composition and a second composition, each composition comprising a drug, where the drug is 5-[4-[2-(N-methyl-N-(2 pyridyl)amino)ethoxy]benzyl]thiazolidine-2,4-dione, or a pharmaceutically acceptable salt or hydrate thereof, and a pharmaceutically acceptable carrier therefor, wherein the first and second compositions are arranged to release the drug at differing release rates on administration such that the rate of release of the drug from the dosage form is substantially independent of pH;
which process comprises the steps of sequentially or simultaneously:
(i) formulating the drug into the first composition; and
(ii) formulating the drug into the second composition;
and the steps of sequentially or simultaneously:
(i) forming the first composition into a first layer; and
(ii) forming the second composition into a second layer; and
(iii) combining the layers into a multilayer dosage form,
whereby the first and second layers are formulated to release drug at differing release rates on administration such that the rate of release of the drug from the dosage form is substantially independent of pH.

30. A process according to claim 29, where, in step (iii), the first and second layers are combined into a bilayer dosage form.

31. A method for the treatment or prophylaxis of a disorder selected from diabetes mellitus, conditions associated with diabetes mellitus, Alzheimer's Disease, mild cognitive impairment, psoriasis, asthma, atherosclerosis, metabolic syndrome, impaired glucose tolerance and impaired fasting glucose, in a human or non-human mammal, which method comprises administering the oral dosage form according to claim 16, to a human or non-human mammal in need thereof.
